(19) United States
(12) Patent Application Publication
(10) Pub. No.: US 2013/0072954 A1
(43) Pub. Date: Mar. 21, 2013

(54) Umbilical Locator

(71) Applicant: Jose C. Suarez, Miami, FL (US)

(72) Inventor: Jose C. Suarez, Miami, FL (US)

(21) Appl. No.: 13/235,363

(22) Filed: Sep. 17, 2011

(51) Int. Cl. A61B 17/34

(52) U.S. Cl. 606/185

(57) ABSTRACT

An umbilical locator that simplifies the localization of the skin flap after it is re-draped over the umbilicus during abdominoplasty surgery. The umbilical locator rests easily and with great stability over the umbilicus while the surgery is performed.

### BACKGROUND

The present invention is an umbilical locator that is used to locate the precise location of the umbilicus during a tummy tuck (abdominoplasty surgery) procedure.

The inventor of the present invention performs abdominoplasty or “tummy tuck” surgery. He performs this cosmetic surgery procedure for patients who are very unhappy with their outer physical abdominal exterior due to the fact that they carry excess fat and/or skin, they have stretch marks, striae, or they have unwanted scars. The surgery involves the removal of excess skin and fat from the lower abdomen, thereby causing the abdomen to have a more cosmetic firm look after the procedure.

Procedures for performing this surgery are as follows: first, incision is made from hip to hip just above the pubic area; then, the abdominal wall is undermined up to the umbilical area; next, incisions are made around the umbilicus to free the navel from the surrounding skin; then the abdominal wall is continued to be undermined up to the xiphoid area. The next part of the procedure involves suturing the rectus abdominal fascia in order to reinforce the integrity of the abdominal wall. The present invention is then used to pinpoint the precise umbilicus position. This is done by lowering the abdominal wall on the present invention after position the invention over the umbilicus. When the abdominal wall is lowered over the present invention, the needle or marker of the present invention marks the exact location of the skin that will cover the umbilicus. Then the physician marks the area to be cut that will be placed over the umbilicus. After marking the area, the present invention is removed and the physician completes the tummy tuck procedure in the classical manner.

The marking of the exact position of the umbilicus position is where some of his colleagues have had difficulties in the past. When they attempted to position the navel to its new location while conducting the procedure, they would rely chiefly on manually finding the umbilicus position; simply because the umbilicus was the normal marker used to locate the exact position of the umbilicus. However, locating the exact position of the umbilical proved problematic, for his colleagues had to reach under the skin flap to feel for it; and even then, they could only get an approximation of where the umbilical was located. Aesthetic and natural appearance are key factors to the end result of abdominoplasty surgery. If not done properly, the belly button will be misaligned and the surgery's aesthetic factors will be reduced.

The inventor was aware of a cone shaped device in the cosmetic surgery industry that claimed to provide a solution for locating the umbilicus. This device was sutured over the umbilical to help define its location. However, after the skin flap was re-draped, it still required the surgeon to reach in, cut its sutures, take it out along with any suture, and then still place the patient's belly button in the correct location relatively unguided. This cumbersome process often resulted in the same misalignment as the traditional technique, simply because the device required too much movement and work under the skin flap. Another umbilical marking procedure utilized in the cosmetic surgery industry required pre-surgical ink marking and diagramming of the abdominal area, followed by sutures, needles, and threads. This procedure also proved cumbersome, time consuming, and provided only marginal position improvements.

These realizations caused the inventor to recognize a need for a small device that could be easily slipped in and out from under the skin, while still providing accurate localization of the umbilical. The inventor noted that a device that did not require attachment to the abdomen would make this movement possible. He also recognized that a wide base was needed for the device to stand up on top of the umbilical without support from sutures or other forms of attachment. Finally, he envisioned a needle like pointer that could penetrate the skin flap, and thus “mark” the exact location of the umbilical.

The above realizations caused him to first create an umbilical locator that could be easily slipped in and out from under the skin, while providing a pinpoint accurate location of the umbilical. The base was wide enough to stand up unattached with sutures or other connectors. The marking pointer was sharp enough to penetrate the skin flap. The marking pointer was also attenuated enough not to leave a visible mark and to provide an accurate, pin point marking device. Finally, the device was small enough and ergonomically designed to be easily slipped in and out from under the skin flap.

Umbilical locating devices have been utilized in the past; yet none with the present characteristics of the present invention. See www.imibeauty.com/html/umbilical_locator.html, and the American Journal of Cosmetic Surgery, Vol. 24, No. 4, 2007.

For the foregoing reasons, there is an umbilical locator that can be easily slipped in and out from under the skin, while providing a pinpoint accurate location of the umbilicus.

### SUMMARY

The present invention is directed to an umbilical locator that is efficacious in providing a pinpoint accurate marking of the umbilicus during abdominoplasty or “tummy tuck” surgeries; thus resulting in properly positioned belly buttons (navels).

The present invention is an umbilical locator that simplifies the localization of the skin flap that will rest over the umbilicus after it is re-draped over the abdomen during abdominoplasty surgery. The umbilical locator rests easily and with great stability over the umbilicus while the surgery is performed for the following reasons: it easily accepts the drape of skin; it punctures a pin size hole through the skin to mark exact umbilical location; and it slips out with one deft move of the surgeons hand after the skin flap is properly positioned over the umbilicus. The present invention is safe, disposable, it may be made of a sterile material, and can easily be used by any surgeon performing an abdominoplasty procedure.

The umbilical locator comprises of a surgically appropriate nonlatex plastic or metal base that is wide enough to rest firmly on the abdomen during surgery; the base may have a length of at least four inches and a width of at least one inch. However other embodiments of this invention could utilize smaller dimensions for the base. The umbilical locator further comprises of a needle. The needle serves as a marker. The needle attaches perpendicularly to the base. In all embodiments of the present invention, the umbilical locator is placed over the umbilicus and pierces a layer of fatty skin draped over it. This provides a visual marker of the umbilicus. The needle of the umbilical locator is long and sharp enough to penetrate the fatty skin flap incised during surgery.

An object of the present invention is to provide an umbilical marking device that, during abdominoplasty surgery, simplifies and makes more efficacious the surgeon's accurate positioning of the skin flap over the umbilicus.

Another object of this invention is to provide a clear and accurate localization of the umbilicus under the re-draped skin flap during abdominoplasty surgery.

Another object of the present invention is to facilitate the localization of the umbilicus during abdominoplasty surgery.

### DRAWINGS

These and other features, aspects, and advantages of the present invention will become better understood with regard to the following description, appended claims, and drawings where:

FIG. 1 is a perspective view of the present invention; and

FIG. 2 is another perspective view of the present invention wherein the needle is attached to the base.

### DESCRIPTION

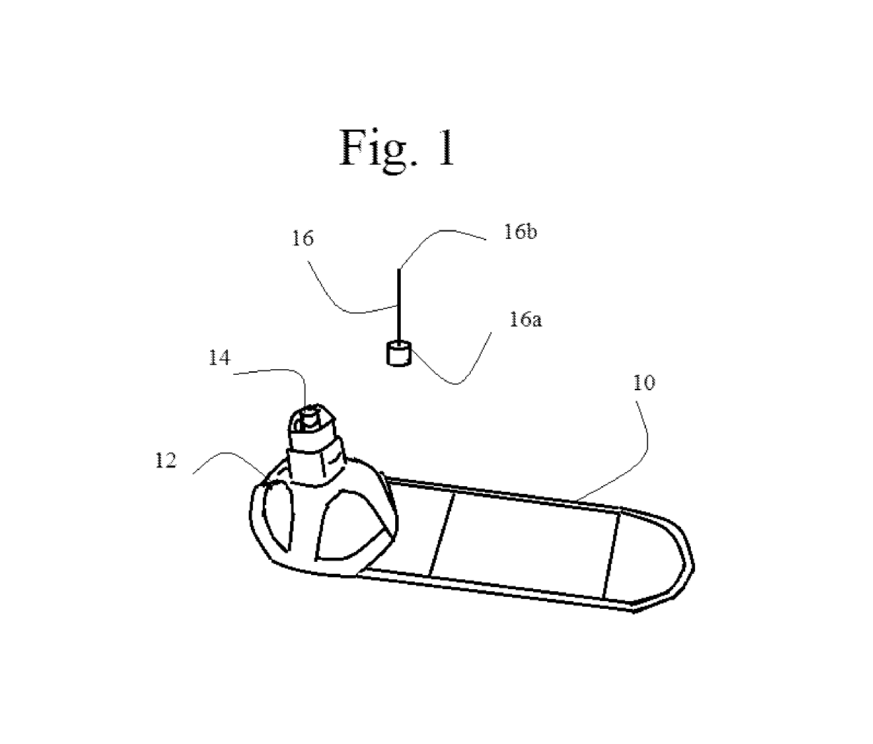

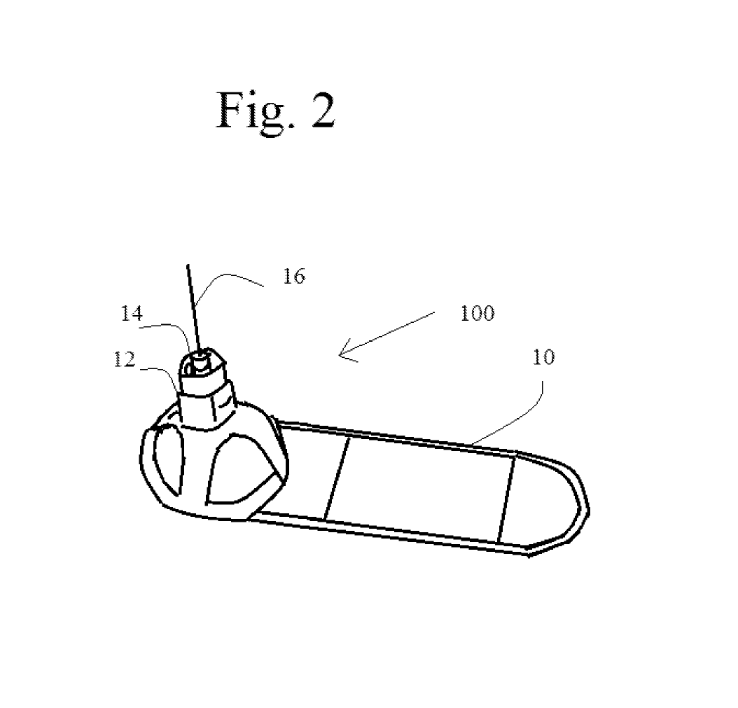

One embodiment of the umbilical locator 100 of the present invention is illustrated in FIGS. 1-2. The umbilical locator 100 is used to provide an exact location of the umbilicus during abdominoplasty surgery. The umbilical locator 100 comprises of a base 10 having a substantially planer body, the base 10 defines a base hub 12 that rises from one of the ends of the substantially planner body 10, the base hub 12 defines a needle receiving adapter 14. The umbilical locator 100 further comprises of a needle 16, the needle 16 has two ends, the first end 16a defines a needle hub 16a and the second end defines a pinpoint 16b, the needle hub 16a attaches to the needle receiving adapter 14.

In an embodiment of the umbilical locator 100, the needle 16 might be at least one inch in length. The needle 16 may be a pin.

The base 10 of the present invention can be made of materials that can be placed in an autoclave for sterilization or may be disposable.

The base 10 of the present invention might be made of the following materials: stainless steel, aluminum, steel, ceramic, plastic, polymers, or of a disposable plastic.

The present invention is used during abdominoplasty surgery. The process of using the invention was previously described in this paper.

An advantage of the present invention is that it facilitates the localization of the umbilicus during an abdominoplasty surgery.

Yet another advantage of the present invention is that it allows the surgeon to place an accurate umbilical marker under the skin and retrieve it prior to the final sewing of the skin in an efficient and fast manner.

Thus the reader will see that at least one embodiment of the umbilical locator 100 provides a more efficient solution for locating the umbilicus during abdominoplasty surgery.

While the inventor's above description contains many specificities, these should not be construed as limitations on the scope, but rather as an exemplification of several preferred embodiments thereof. Many other variations are possible. For example, the marker does not necessarily have to be a needle like device, it could be also utilize lasers or unique fibers to perform the marking function. Accordingly, the scope should be determined not by the embodiments illustrated, but by the appended claims and their legal equivalents.

What is claimed is:

1. An umbilical locator for providing an exact location of the umbilicus during an abdominoplasty surgery, the umbilical locator comprises of:
a base having a substantially planer body, the base defines a base hub that rises from one of the ends of the substantially planer body, the base hub defines a needle receiving adapter; and
a needle, the needle having two ends, the first end defines a needle hub and the second end defines a pinpoint, the needle hub attaches to the needle receiving adapter.

2. The umbilical locator of claim 1, wherein the needle is at least one quarter of an inch in length.

3. The umbilical locator of claim 2, wherein the base is made of a material that can be placed in an autoclave.

4. The umbilical locator of claim 3, wherein the material is stainless steel.

5. The umbilical locator of claim 3, wherein the material is ceramic.

6. The umbilical locator of claim 3, wherein the material is a metal.

7. The umbilical locator of claim 2, wherein the base is made of a disposable plastic material.
